(19) United States
(12) Patent Application Publication
(10) Pub. No.: US 2025/0276216 A1
(43) Pub. Date: Sep. 4, 2025

(54) GENERATIVE-AI ENHANCED KINEMATIC DATA MEASUREMENT SYSTEM

(71) Applicant: Stryd, Inc., Boulder, CO (US)

(72) Inventors: Wyatt Mohrman, Boulder, CO (US); Kun Li, Broomfield, CO (US); James Williamson, Broomfield, CO (US)

(73) Assignee: Stryd, Inc., Boulder, CO (US)

(21) Appl. No.: 19/068,684

(22) Filed: Mar. 3, 2025

Related U.S. Application Data
(60) Provisional application No. 63/561,150, filed on Mar. 4, 2024.

(51) Int. Cl. A63B 24/00; A61B 5/00; A61B 5/11; G01C 19/5776; G01P 15/18

(52) U.S. Cl. CPC A63B 24/0075; A61B 5/112; A61B 5/6829; A61B 5/7267; A61B 5/7282; G01C 19/5776; G01P 15/18

(57) ABSTRACT

The technology relates to measurement systems and methods for measure kinematic data of an athlete during athletic activities. In an example, the system includes a housing that includes an accelerometer and a gyroscope; at least one processor; and at least one memory. The system performs operations including generating kinematic data for the athlete during the athletic activity; detecting an occurrence of a feedback trigger condition; surfacing a feedback request for a natural language response from the athlete during the athletic activity; receiving a natural language response from the athlete during the athletic activity; generating an athlete-response prompt for a generative artificial intelligence (AI) model; providing the athlete-response prompt as input to the generative AI model; receiving, from the generative AI model in response to the athlete-response prompt, a standardized tag for the defined categories; and tagging the kinematic data with the standardized tag.

### CROSS-REFERENCE TO RELATED APPLICATIONS

This application claims the benefit of U.S. Provisional Application No. 63/561,150 filed on Mar. 4, 2024, the disclosure of which is herein incorporated by reference in its entirety. To the extent appropriate, a claim of priority is made to the application.

### BACKGROUND

As athletes perform physical activities, such as running, many factors may affect the performance of the athlete. Such factors may be internal or external factors. For instance, internal changes, such as injury or health, may affect the performance of the athlete. External changes, such as heat, may also affect the performance of the athlete. It is with respect to these and other considerations that examples of the technology discussed herein have been made. In addition, although relatively specific problems have been discussed, it should be understood that the examples should not be limited to solving the specific problems identified in the background.

### SUMMARY

This summary is provided to introduce a selection of concepts in a simplified form that are further described below in the Detailed Description section. This summary is not intended to identify key features or essential features of the claimed subject matter, nor is it intended as an aid in determining the scope of the claimed subject matter.

In an aspect, the technology relates to a measurement system that is retained by an athlete during an athletic activity. The measurement system includes a housing that includes an accelerometer and a gyroscope; at least one processor; and at least one memory, the at least one memory storing instructions that, when executed by the at least one processor cause the system to perform operations. The operations include generate kinematic data for the athlete during the athletic activity based on measurements from the accelerometer and the gyroscope; detect an occurrence of a feedback trigger condition; based on the occurrence of the feedback trigger condition, surface a feedback request for a natural language response from the athlete during the athletic activity; receive, in response to the feedback request, a natural language response from the athlete during the athletic activity; generate an athlete-response prompt for a generative artificial intelligence (AI) model, the athlete-response prompt including the natural language response and instructions to transform the natural language response to at least one standardized tag for one or more defined categories; provide the athlete-response prompt as input to the generative AI model; receive, from the generative AI model in response to the athlete-response prompt, an output payload includes the at least one standardized tag for the one or more defined categories; and tag the kinematic data with the at least one standardized tag for the one or more defined categories to create tagged kinematic data.

In an example, the operations further comprise train a machine learning (ML) model the tagged kinematic data. In a further example, the kinematic data is first kinematic data and the athletic activity is a first athletic activity, and the operations further include generate second kinematic data for the athlete during a second athletic activity based on measurements from the accelerometer and the gyroscope; provide the second kinematic data as input to the trained ML model during the second athletic activity; and receive, as output from the trained ML model during the second athletic activity, a standardized classification of the kinematic data. In a still further example, the operations further include, based on the standardized classification of the kinematic data, surface a notification to the athlete. In another example, the operations further include, based on the standardized classification of the kinematic data, adjust at least one of a pacing target or routing target for the athlete during the second athletic activity. In yet another example, the operations further include, based on the standardized tag, adjusting a pacing target for the athlete during the athletic activity. In still another example, the operations further include generating the feedback request by: generate an initial-request prompt instructing the generative AI model to generate the feedback request; provide the initial-request prompt as input to the generative AI model; and receive, from the generative AI model in response to the initial-request prompt, an output payload with the feedback request. In a further example, the initial-request prompt includes at least one of user-profile data for the athlete or the kinematic data.

In another aspect, the technology relates to a computer-implemented measurement method for measuring kinematic data of an athlete during athletic activities. The method includes generating kinematic data for the athlete during an athletic activity based on measurements from at least one of an accelerometer and a gyroscope; detecting an occurrence of a feedback trigger condition; based on the occurrence of the feedback trigger condition, surface a feedback request for a natural language response from the athlete during the athletic activity; receiving, in response to the feedback request, a natural language response from the athlete during the athletic activity; generating an athlete-response prompt for a generative artificial intelligence (AI) model, the athlete-response prompt including the natural language response and instructions to transform the natural language response to at least one standardized tag for one or more defined categories; providing the athlete-response prompt as input to the generative AI model; receiving, from the generative AI model in response to the athlete-response prompt, an output payload includes the at least one standardized tag for the one or more defined categories; and tagging the kinematic data with the at least one standardized tag for the one or more defined categories to create tagged kinematic data.

In an example, the method further includes, based on the standardized tag, performing at least one of: adjusting a pacing target for the athlete during the athletic activity; or surfacing a notification to the athlete. In another example, the kinematic data is first kinematic data and the athletic activity is a first athletic activity, and the method further includes: training a machine learning (ML) model the tagged kinematic data; generating second kinematic data for the athlete during a second athletic activity based on measurements from the accelerometer and the gyroscope; providing the second kinematic data as input to the trained ML model during the second athletic activity; and receiving, as output from the trained ML model during the second athletic activity, a standardized classification of the kinematic data. In yet another example, the method further includes, based on the standardized classification, adjusting a pacing target for the athlete during the second athletic activity. In still another example, the standardized classification is for at least one of a health category or an injury category. In yet another example, the method further includes based on the standardized classification, surfacing an alert to the athlete indicating an injury or health condition of the athlete. In still yet another example, the method further includes generating the feedback request by: generating an initial-request prompt instructing the generative AI model to generate the feedback request; providing the initial-request prompt as input to the generative AI model; and receiving, from the generative AI model in response to the initial-request prompt, an output payload with the feedback request. In another example, generating the kinematic data and detecting the occurrence of a feedback trigger condition are performed by a foot pod housing the accelerometer and the gyroscope.

In another aspect, the technology relates to a computer-implemented measurement method for measuring kinematic data of an athlete during athletic activities. The method includes generating kinematic data for the athlete during an athletic activity based on measurements from at least one of an accelerometer and a gyroscope; providing the kinematic data at input to a trained machine learning (ML) model, wherein the trained ML model is trained based on prior kinematic data tagged with standardized tags generated from a generative artificial intelligence (AI) model based on natural language responses received from one or more athletes; receiving, as output from the trained ML model during the athletic activity, a standardized classification of the kinematic data; and based the standardized classification adjusting a target for the athlete during the athletic activity.

In an example, the standardized classification indicates the athlete is suffering at least one of an injury or a health condition, and adjusting the pacing target adjusts the pacing target to a slower pace. In another example, the method further includes, based on the standardized classification, surfacing an alert to the athlete. In still another example, the method further includes receiving, in response to the alert, and input from the athlete; and performing a reinforcement training of the ML model based on the input from the athlete.

The details of one or more aspects are set forth in the accompanying drawings and description below. Other features and advantages will be apparent from a reading of the following detailed description and a review of the associated drawings. It is to be understood that the following detailed description is explanatory only and is not restrictive of the invention as claimed.

### BRIEF DESCRIPTION OF THE DRAWINGS

The accompanying drawings, which are incorporated in and constitute a part of this disclosure, illustrate various aspects of the present invention. In the drawings:

FIG. 1A depicts a perspective view of a measurement platform in the form of a foot pod attached to a shoe.

FIG. 1B depicts an exploded view of the foot pod of FIG. 1A.

FIG. 2 depicts an example measurement platform.

FIG. 3 depicts an example performance measurement system according to examples of the present disclosure.

FIG. 4 depicts an example method for tagging kinematic data with objective feedback data.

FIG. 5 depicts an example method for classifying kinematic data.

### DETAILED DESCRIPTION

The following detailed description refers to the accompanying drawings. Wherever possible, the same reference numbers are used in the drawing and the following description to refer to the same or similar elements. While aspects of the invention may be described, modifications, adaptations, and other implementations are possible. For example, substitutions, additions, or modifications may be made to the elements illustrated in the drawings, and the methods described herein may be modified by substituting, reordering, removing, or adding stages or operations to the disclosed methods. Accordingly, the following detailed description does not limit the invention, but instead, the proper scope of the invention is defined by the appended claims. Examples may take the form of a hardware implementation, or an entirely software implementation, or an implementation combining software and hardware aspects. The following detailed description is, therefore, not to be taken in a limiting sense.

As briefly discussed above, internal and external factors can affect the performance of an athlete. While estimates of such factors may be possible from transducer-based sensors, the actual perceived effect of such factors on the athlete may continue to be difficult to discern. For instance, when a performance degradation or change is measured for an athlete's performance, the underlying reasons may continue to be difficult to determine. As an example, a temperature sensor may indicate a particularly high environmental temperature, but other factors may also, or alternatively, be contributing to a change in performance of the athlete, such as health, nutrition, or injury of the athlete.

The present technology provides for systems and methods that are capable of capturing measures of factors affecting the athlete based on natural language feedback acquired from the athlete. For example, during a physical activity, a sensor platform may identify a degradation or change in the performance of the athlete. As a result, feedback may be requested from the athlete, during the physical activity, as to the subjective feelings of the athlete. This feedback request may be provided in the form of an audio message, and the response from the athlete may similarly be received in an audio format. The natural language feedback that is received from the athlete is then processed by a generative artificial intelligence (AI) model (e.g., large language model (LLM)) to form an objective measure or score for the feedback. In some instances, the generative AI model also generates the request for the feedback. The kinematic data measured from the sensor platform may then be tagged with the objective measures from the generative AI model.

In some examples, the tagged kinematic data may also be used to train a machine-learning (ML) model to classify later live kinematic data, without the need for further feedback requests from the user. For example, once the ML model is trained, the kinematic data can be classified based on the object measures for which the model is trained. Accordingly, additional factors potentially affecting the athlete can be determined from the kinematic data alone through the use of the trained ML model. As a result, the present system is able to effectively provide a measurement system for subjective states of the athlete.

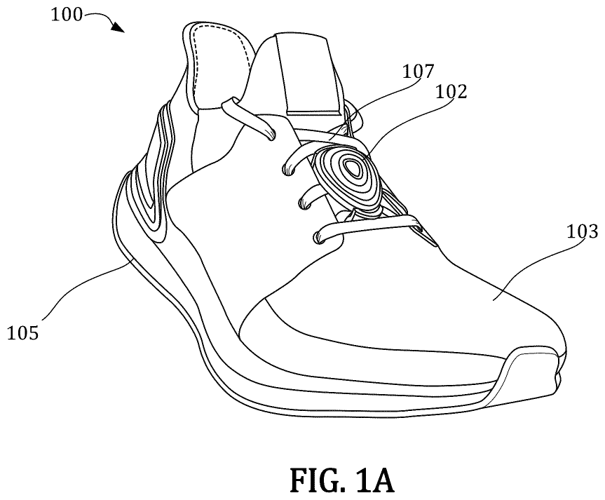

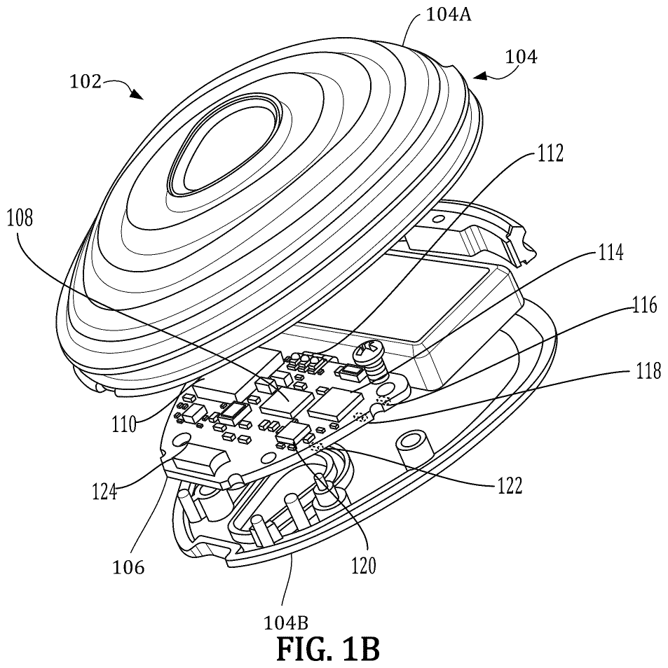

FIG. 1A depicts a perspective view of a measurement platform 100 in the form of a foot pod 102 attached to a shoe 103. As discussed further below, the foot pod 102 may house measurement components that allow for kinematic data to be generated for the athlete. By attaching the foot pod 102 and the sensors therein to the shoe 103 of the athlete, higher integrity data may be obtained about the movement of the foot of the athlete.

In the example depicted, the foot pod 102 attaches to the exterior of the shoe 103. More specifically, the foot pod 102 attached to the laces 107 of the shoe 103. The foot pod 102 may include a detachable clip that can be securely clipped to the laces. In other examples, the foot pod 102 may be attached to the exterior of the shoe 103 in other manners or be attached to different portions of the shoe 103. For instance, the foot pod 102 (or the components thereof) may be integrated into the shoe 103. As an example, the components of the foot pod 102 may be integrated into the sole 105 of the shoe 103, among other locations. While the foot pod 102 is shown as connected to the shoe 103, in other examples the foot pod 102 may be a worn pod that can be clipped or connected to other parts of the athlete or the athlete's clothing (e.g., shorts or shirt).

FIG. 1B depicts an exploded view of the foot pod 102 of FIG. 1A. The example foot pod 102 includes a housing 104 composed of a top shell 104A and a bottom shell 104B that connects to the top shell 104A to form the housing 104. The housing 104 defines an inner cavity that houses a plurality of computing and measurement components of the measurement platform 100. The computing and measurement components may be included on a circuit board 106 within the housing 104. For instance, the measurement components may include at least a processor 108, memory 110, multiple accelerometers 112, 114, 116, a gyroscope 118, a magnetometer 120, environmental sensors 122, and a radio-frequency (RF) antenna 124. The environmental sensors 122 may include sensors such as temperature, pressure, and/or humidity sensors.

The multiple accelerometers 112-116 may each be three-axis accelerometers that generate multi-axis motion data. The first accelerometer 112 may provide a wake function that creates a wake signal upon movement to activate the system. The second accelerometer 114 may provide a high-dynamic range signal, and the third accelerometer 116 may provide a low-dynamic range signal. In other examples, only a single accelerometer may be used. The gyroscope 118 may be used to generate orientation data. The orientation data in combination with the motion data from the accelerometer(s) may be used to generate additional metrics as discussed herein. The magnetometer 120 may also generate orientation data, and in some examples, either the gyroscope 118 or the magnetometer 120 may be omitted. The combination of an accelerometer with a gyroscope and/or a magnetometer may be referred to herein as an inertial measurement unit (IMU) that generates or outputs multi-axis motion data. The IMU and/or components thereof are used to generate kinematic data (e.g., multi-axis motion data) that represents the motion of the athlete, or limb/portion of the athlete (e.g., leg, foot), during an athletic activity.

The processor 108 may be a microprocessor, such as a central processing unit (CPU). In other examples, the processor 108 may be a field-programmable gate array (FPGA) or an applications-specific integrated circuit (ASIC). The memory 110 may include volatile storage (e.g., random access memory), non-volatile storage (e.g., read-only memory), flash memory, or any combination of such memories. The memory 110 may include instructions that, when executed by the processor 108, cause the example foot pod 102 to perform operations discussed herein. For instance, local processing of the signals from the IMU may be performed on the foot pod to generate the kinematic data. In addition, local processing of the kinematic data may also be performed, such as to determine changes or trends in the kinematic data.

The radio-frequency (RF) antenna 124 may be used to communicate data from the foot pod 102 and/or received data at the foot pod 102. For instance, the kinematic data may be communicated from the foot pod 102 to a secondary device, such as a smartphone or watch that is carried or worn by the athlete during the athletic activity.

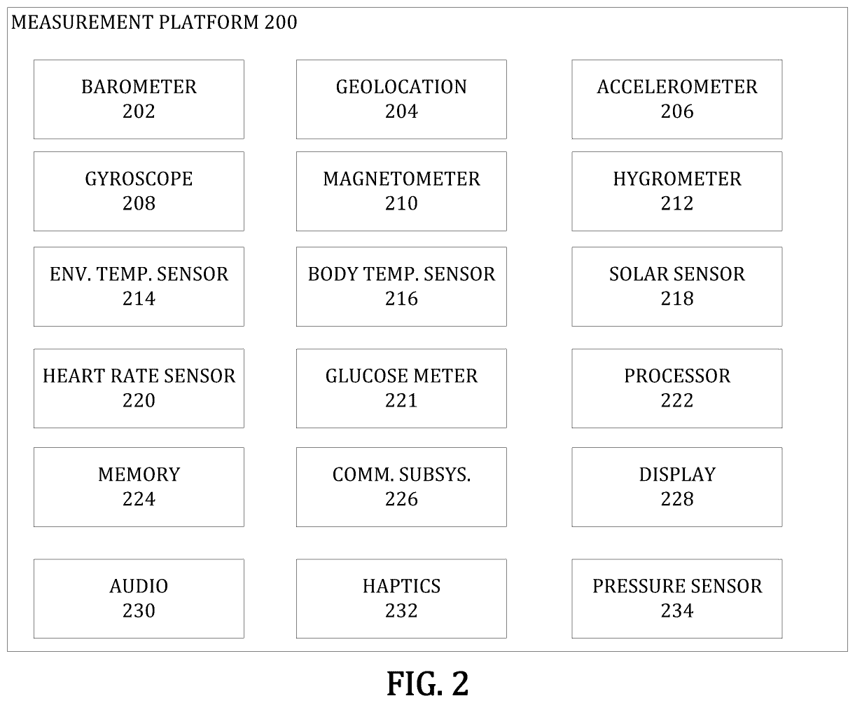

FIG. 2 depicts an example measurement platform 200. The example measurement platform 200 may be similar to the measurement platform 100 discussed above. The example measurement platform includes a barometer 202, a geolocation receiver 204, an accelerometer 206, a gyroscope 208, a magnetometer 210, a hygrometer 212, an environmental temperature sensor 214, a body temperature sensor 216, a solar sensor 218, a heart rate sensor 220, a glucose meter 221, a processor 222, memory 224, a communications subsystem 226, a display 228, audio output 230, haptics 232, and/or a pressure 234. In some examples, not all of the computing and measurement components of the measurement platform 200 may be included, and in some examples additional computing and measurement components are included in the measurement platform 200.

In some examples, the components of the measurement platform 200 may be distributed across multiple distributed devices of the athlete. For example, the athlete may be wearing one or more wrist-based devices, such as a sports watch and/or a smartwatch. The athlete may also or alternatively be wearing a chest-strap device and/or have a smartphone that is attached, held, or otherwise retained by the athlete.

The various computing and measurement components of the measurement platform 200 may be distributed in different manners across the devices of the athlete, such as any worn device of the athlete and/or retained by the athlete while performing the athletic activity. For instance, the feedback components may be incorporated into a smartphone and/or a watch. The remaining components may then be housed in a foot pod 102, another wearable/clipped pod, a chest strap device, and/or integrated into the shoe itself or a portion of shoe, such as an insole. Each of the devices may have its own housing that houses one or more of the components of the measurement platform. In some examples, all the components of the measurement platform 200 are incorporated into a single device, such as a watch or pod 102.

The measurement platform 200 may also be distributed across different housings that are retained by or attached to the athlete (via straps or clothing). For instance, the housings may be on the front, back, or side of the torso, on the front, back, or side of the waist, embedded in clothing, attached to or embedded within one or both shoes, on one or both socks, on the front or back of a headband, on one or both thighs, in one or both arm bands, or on one or both calves. Depending upon the implementation, each location can have one or more of the following advantages: convenience to the user, accuracy of measuring running and/or walking technique and distance, accuracy in measuring the geographic position of the user, improved sensed data quality, improved user comfort, monitoring of one or more limbs of a user; and reduced stress on the sensing device. By way of example, beneficial locations can include within or on a strap attached to the ankle, leg, wrist, waist, or torso. The sensor platform, or portions thereof, can also be placed within or on apparel such as clothing, belts, or shoes. It can also be placed on, under, or to the side of the foot.

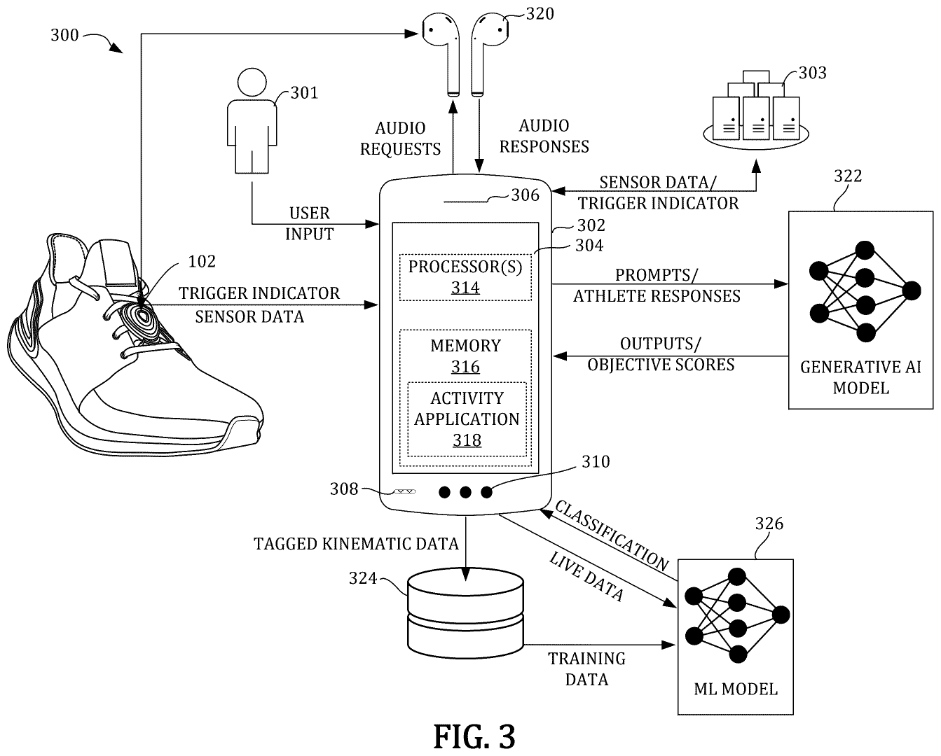

FIG. 3 depicts an example performance measurement system 300 according to examples of the present disclosure. In the example depicted, the system 300 includes the foot pod 102, a portable computing device 302, headphones 320, a generative AI model 322, a kinematic database 324, and an ML model 326. In some examples, the foot pod 102 may be replaced by the measurement platform 200 and/or a combination of components thereof. The system may also receive interactions and/or user inputs from a user 301. One or more remote servers 301, such as a cloud-based server 301, may also be utilized by or incorporated into the system 300.

The example computing device 302 depicted includes a display screen 304 (which may be a touchscreen), a speaker 306, a microphone 308, and one or more input elements 310 (e.g., buttons or keys). The computing device 302 also includes additional computing components, such as one or more processors 314 and memory 316. The memory 316 stores instructions and/or programs that, when executed, by the one or more processors 314 cause the computing device 302 to perform the operations discussed herein. For example, the memory 316 may store an activity application 318 that causes the computing device 302 to perform the functions and operations described herein. The portable computing device 302 may also include further communication components, such as wireless communication components (e.g., radio frequency, Bluetooth, Wi-Fi) that allow for communication to occur between the pod 102 and the other components of the example performance measurement system 300, such as the generative AI model 322 and the ML model 326. While the portable computing device is depicted as a smartphone, in other examples, the portable computing device 302 may be another type of device that is worn or carried by the athlete during performance of the physical activity, such as a smartwatch and/or wearable smart glasses or other smart devices.

The generative AI model 322 may be a variety of types of generative AI models, such as a language model (LM), a large language model (LLM), a multimodal model, or other type of generative AI model. Example models may include the GPT models from OpenAI, BARD from Google, and/or LLaMA from Meta, among other types of generative AI models. The generative AI model 322 may operate on a remote device, such as a cloud server (e.g., cloud server 303 or a different cloud server), that is remote from the portable computing device 302. Accordingly, the portable computing device 302 may communicate with the generative AI model 322 via an Internet connection. In other examples, the generative AI model 322 may operate on the portable computing device 302.

According to example implementations, the generative AI model 322 is trained to understand and generate sequences of tokens, which may be in the form of natural language (e.g., human-like text). In various examples, the generative AI model 322 can understand complex intent, cause and effect, perform language translation, semantic search classification, complex classification, text sentiment, summarization, summarization for an audience, and/or other natural language capabilities.

In some examples, the generative AI model 322 is in the form of a deep neural network that utilizes a transformer architecture to process the text it receives as an input or query. The neural network may include an input layer, multiple hidden layers, and an output layer. The hidden layers typically include attention mechanisms that allow the generative AI model 322 to focus on specific parts of the input text, and to generate context-aware outputs. The generative AI model 322 is generally trained using supervised learning based on large amounts of annotated text data and learns to predict the next word or the label of a given text sequence.

The size of a generative AI model 322 may be measured by the number of parameters it has. For instance, as one example of an LLM, the GPT-4 model from OpenAI has billions of parameters. These parameters may be weights in the neural network that define its behavior, and a large number of parameters allows the model to capture complex patterns in the training data. The training process typically involves updating these weights using gradient descent algorithms, and is computationally intensive, requiring large amounts of computational resources and a considerable amount of time. The generative AI model 322 in examples herein, however, is pre-trained, meaning that the generative AI model 322 has already been trained on the large amount of data. This pre-training allows the model to have a strong understanding of the structure and meaning of text, which makes it particularly effective for the specific tasks discussed herein.

The generative AI model 322 may operate as a transformer-type neural network. Such an architecture may employ an encoder-decoder structure and self-attention mechanisms to process the input data (e.g., a prompt). Initial processing of the prompt may include tokenizing the prompt into tokens that may then be mapped to a unique integer or mathematical representation. The integers or mathematical representations combined into vectors that may have a fixed size. These vectors may also be known as embeddings.

The initial layer of the transformer model receives the token embeddings. Each of the subsequent layers in the model may uses a self-attention mechanism that allows the model to weigh the importance of each token in relation to every other token in the input. In other words, the self-attention mechanism may compute a score for each token pair, which signifies how much attention should be given to other tokens when encoding a particular token. These scores are then used to create a weighted combination of the input embeddings.

In some examples, each layer of the transformer model comprises two primary sub-layers: the self-attention sub-layer and a feed-forward neural network sub-layer. The self-attention mechanism mentioned above is applied first, followed by the feed-forward neural network. The feed-forward neural network may be the same for each position and apply a simple neural network to each of the attention output vectors. The output of one layer becomes the input to the next. This means that each layer incrementally builds upon the understanding and processing of the data made by the previous layers. The output of the final layer may be processed and passed through a linear layer and a softmax activation function. This outputs a probability distribution over all possible tokens in the model's vocabulary. The token(s) with the highest probability is selected as the output token(s) for the corresponding input token(s).

The ML model 326 may be in the form of a model that may be trained using supervised learning techniques. For instance, the model may be in the form of a logistic regression model, a decision tree, a random forest, a support vector machine, a neural network, a k-nearest neighbors model, and/or a naïve Bayes model, among others. The ML model 326 may operate on a remote device, such as a cloud server, that is remote from the portable computing device 302. Accordingly, the portable computing device 302 may communicate with the ML model 326 via an

Internet connection. The ML model 326 may be a model that is significantly smaller than the generative AI model 322, such that the ML model 326 requires fewer processing and/or memory resources to process inputs. Due to the smaller size of the ML model 326, the ML model may also be stored and executed on the pod 102 and/or of the portable computing device 302 in some examples.

The kinematic database 324 may be a variety of different types of databases or data stores. As discussed further below, the kinematic database 324 receives and stores tagged kinematic data based on sensor measurements and feedback received from the athlete during the physical activity. The tagged kinematic data in the kinematic database 324 may then be used to train the ML model 326. The kinematic database 324 may be stored on a server or computing device that is remote from the example computing device 302, such as a cloud server (e.g., cloud server 303 or another server). In some examples, the kinematic database 324 may be stored on the same server or device as the ML model 326.

During operation, the pod 102 generates kinematic data through its various sensors, such as the IMU. The measured kinematic data may be processed locally by the pod 102. Processing of the kinematic data may be performed to identify changes from a baseline of physical activity for a user. The changes may be indicative of a performance degradation for the athlete during the physical activity. Such a change may cause the pod 102 to issue a trigger indicator to the portable computing device 302 (e.g., to the activity application 318). The trigger indicator indicates that the portable computing device 302 is to request feedback from the athlete during the physical activity.

In other examples, the kinematic data and/or other sensor data measured by the pod 102 may be additionally or alternatively transmitted to the portable computing device 302. In such examples, the portable computing device 302 may analyze the received data to determine the change in performance. The portable computing device 302 may then locally triggers the feedback requests. In still further examples, the kinematic data and/or other sensor data measured by the pod 102 may be transmitted to the cloud server 303. In such examples, the cloud server 303 may analyze the received data to determine the change in performance. The server 303 may then transmit the trigger indicator to the portable computing device.

In still other examples, a user input may be received by the portable computing device 302 from the user 301 that indicates the feedback acquisition process should be initiated. For instance, as the user 301 is running or performing the athletic activity, the user 301 may realize that something is unusual or a particular condition is occurring. In such cases, the user 301 may provide a trigger input or indicator to trigger the feedback acquisition process. The user input may in the form of a touch input or manual input. In other examples, the user input may be a speech or audible request detected via a microphone.

In response to receiving or generating the trigger indicator, the portable computing device 302 begins a feedback acquisition process. In an example, the feedback acquisition process includes generating a prompt for the generative AI model 322. The prompt is an input sequence that typically includes text data but may also include other modes of input (e.g., audio and/or image data). The prompt itself may be a discrete data structure including the data and/or elements discussed herein. The prompt requests that the generative AI model 322 generate a request for feedback from the athlete. The request may be in the form of a question or statement that is intended to elicit a natural language response from the athlete. The prompt may further include a number of defined categories for which the generative AI model 322 is to elicit feedback. For instance, such categories may relate to health, injury, hydration, weather, or mood, among other categories.

The prompt may further include data about the athlete and/or the physical activity that is currently being performed by the athlete. For instance, the prompt may be dynamically populated with sensor data (e.g., the kinematic data or a portion thereof) that is received from the pod 102. Additionally or alternatively, the prompt may also be dynamically populated with user profile data for the athlete. The user profile data for the athlete may include data such as the athlete's height, weight, and gender. The user profile may also include additional information about the athlete's current state, such as current nutrition or hydration information, sleep information, shoe type, apparel, current injuries, among other information. This initial prompt that instructs the generative AI model 322 to generate the feedback request(s) may be referred to as an initial-request prompt.

The initial-request prompt is then provided as input to the generative AI model 322. The generative AI model 322 processes the received prompt to generate an output payload that includes the request for feedback from the athlete. The generated request for feedback is then received by the portable computing device 302 from the generative AI model 322.

In other examples, the request for feedback may be accessed or generated locally on the portable computing device 302. For instance, the request for feedback may be based on stored templates and/or questions for the athlete. In some examples, a particular question and/or stored template may be selected based on the type of activity that is being performed by the athlete, data in the user profile of the athlete, and/or sensor data received from the pod 102.

Local generation or local access of questions allows for feedback functionality even in scenarios where the portable computing device 302 does not have a connection to the internet, such as when the physical activity is taking place in a location without wireless internet or cellular phone connectivity. In contrast, having the request for feedback be generated by the generative AI model 322 requires connectivity to the generative AI model 322 (often through the internet), but the generative AI model 322 may be able to generate more robust requests and enable a chat-session to occur effectively between the athlete and the generative AI model 322. The chat session allows for the ability for the questions to adapt to prior answers and increases the likelihood of receiving meaningful feedback from the athlete that can be used for the purposes described herein.

After receiving or accessing the request for feedback, the generated request is surfaced to the athlete during the physical activity. The request may include, in some examples, a plurality of questions that are presented in a series to the athlete. For instance, the questions regard the different categories discussed herein and may request the subjective feedback from the athlete. As examples, questions may relate to the athlete currently feels, if they are feeling injured or fatigued, details about the injury or fatigue, if the athlete is thirsty or hungry, if the athlete is hot or cold, or shoe characteristics (e.g., if the shoe feels supportive, responsive, stable), among other types of questions. The questions may be surfaced by displaying the questions of the display screen 304 of the portable computing device 302. In other examples the questions may be surfaced via the speaker 306 and/or headphones 320 that are connected (wirelessly or wired) to the portable computing device 302. In examples where the questions are presented in an audio format, a text-to-speech operation may be performed on the questions that are received and/or accessed in a text format.

Responses to the request for feedback (e.g., questions) are then received from the athlete. In some examples, the response is provided as an audio response that includes a free-form narrative response from the athlete in natural language. The responses may be received via the microphone 308 on the portable computing device 302 and/or through a microphone that is built into the headphones 320. The audio input may then be transformed to text via a speech-to-text function of the portable computing device 302. In some examples, some of the speech processing and communication may be performed by the processing components of the pod 102. In such examples, the pod 102 may have a direct wireless connection with the headphones 320 or other similar audio device to facilitate connections directly between the pod 102 and the headphones 320.

In other examples, the responses are received through interactions with the touchscreen display 304 and/or the input elements 310. Such responses may still be in narrative, natural language form. In some instances, the surfaced request for feedback may include multiple-choice options to for the user to provide the response(s). The response from the user may then be a selection of one or more of those multiple-choice options. The multiple-choice options may be a different set of narrative answers. The particular multiple-choice options may also be based on the data included in the user profile, the sensor data, and/or the activity being performed. For instance, in examples where the request for feedback is generated by the generative AI model 322 in response to the initial-request prompt, the questions and multiple-choice responses (if present) are based on the data provided in the initial-request prompt.

The responses that are received by the portable computing device 302 may be immediately provided to the generative AI model 322, such as when the generative AI model 322 is supporting a chat session between the athlete and the generative AI model 322. Alternatively or additionally, the responses may be stored locally by the portable computing device 302.

Once the responses to all the requests/questions have been received, the responses may be incorporated into another prompt that is provided as input to the generative AI model 322. For instance, in examples where the requests for feedback are generated locally due to a lack of an internet connection, the responses may be incorporated into the prompt and provided to the generative AI model 322 when an internet connection is established. This prompt including the athlete responses may be referred to herein as the athlete-response prompt.

In addition to the dynamically populated response data, the athlete-response prompt also includes instructions that instruct the generative AI model 322 to transform the narrative responses from the athlete into an objective scores for one or more categories. For instance, the objective scores may range from 0-10 or some other scale. The categories may be the types of categories discussed herein, such as health, injury, fatigue, hydration, weather, athletic equipment (e.g., shoes), or mood, among other categories. Due to the deep contextual understanding that is provided by the generative AI model 322, such a tool is particularly well suited for this particular subjective-to-objective translational task. In response to the athlete-response prompt, the generative AI model 322 processes the athlete-response prompt and generates an output payload that includes standardized tags (e.g., the objective scores) for the categories (e.g., categories that were identified in the athlete-response prompt).

In some examples, the athlete-response prompt is not separately provided from the initial-request prompt. Rather, the initial-request prompt may include instructions that the responses that are received by the athlete are to be translated into the standardized tags (e.g., objective scores) for the one or more categories. As such, based on the instructions in the initial-request prompt, the generative AI model 322 conducts a chat session between the athlete and the generative AI model 322 to collect the responses from the athlete, and the generative AI model 322 generates the standardized tags based on the subjective responses received from the athlete.

The output payload containing the standardized tags may then be received by the example computing device 302 and/or the kinematic database 324. The objective scores are then correlated with the kinematic data to which the standardized tags (e.g., objective scores) correspond. For instance, the corresponding kinematic data is tagged with the standardized tags from the kinematic database 324. The kinematic data that corresponds to the standardized tags may be a set amount of kinematic data for a time period around the time the responses were received from the athlete. As an example, the corresponding kinematic data may be the kinematic data collected N number of minutes (e.g., less than or equal to 2 minutes, 5 minutes, 10 minutes) prior to the responses being received from the athlete. In other examples, the corresponding kinematic data may be the kinematic data collected N number of minutes before and after the responses being received from the athlete, which allows for correlation of the standardized tags to kinematic data collected before and after the responses were received.

The tagging of the kinematic data with the standardized tags (e.g., objective scores) may be performed by the computing device 302 and/or by the kinematic database 324 (or device storing the kinematic database 324). The tagged kinematic data is then stored in the kinematic database 324. For example, where the tagging is performed by the computing device 302, the tagged kinematic data is transmitted to the kinematic database 324 for storage in the kinematic database 324. In examples, where the kinematic data is tagged by the kinematic database 324, the kinematic data may be transmitted separately from the objective scores. For instance, the kinematic database 324 may receive the kinematic data from the example computing device 302 and receive the objective scores from the generative AI model 322.

In examples, the tagged kinematic data is then used to train the ML model 326. The ML model 326 may be trained using supervised training methods. Supervised training uses the tagged kinematic data as the labeled training dataset, with the kinematic data being treated as the input and the standardized tags being treated as the known output or ground-truth output. During training, the weights of the ML model 326 may be iteratively adjusted based on the tagged kinematic data until the ML model 326 is able to accurately predict or classify the kinematic data to match the standardized tags (within an accuracy tolerance).

The ML model 326 may be trained based on tagged kinematic data from multiple athletes and/or based on tagged kinematic data for a specific athlete. As such, the ML model 326 may be athlete-specific (e.g., an individualized model) and/or a more general model that can apply to multiple athletes. In examples where the ML model 326 applies to multiple athletes, the tagged kinematic data in the kinematic database 324 may be received for multiple athletes for multiple athletic activities. The combined kinematic data may then be used for training the ML model 326. In some examples, the kinematic data may be grouped for different groups of athletes, such as athletes grouped by age, gender, height, and/or weight, among other characteristics. Different ML models 326 may then be trained for each group of athletes based on the combined tagged kinematic data for each of the groups of athletes.

Once the ML model 326 is trained, the ML model 326 may be used for classifying and/or evaluating live or real-time kinematic data. As an example, when an athlete is performing an athletic activity, the pod 102 generates real-time kinematic data via the sensors of the pod 102. The real-time kinematic data is provided to the computing device 302, and the example computing device 302 transmits the real-time kinematic data to the ML model 326. The ML model 326 receives the real-time kinematic data as input and processes the real-time kinematic data. In some examples, the ML model 326 may be stored locally on the computing device 302 and/or the pod 102, and the kinematic data may be provided as input to the ML model 326 locally.

The ML model 326 produces an output that includes one or more standardized classifications (e.g., objective scores) for the kinematic data according to the categories for which the ML model 326 was trained. As an example, if the ML model 326 was trained on tagged kinematic data having objective scores for categories of “health” and “injury”, the ML model 326 is able to predict the current objective scores for the health and injury status of the athlete based on the kinematic data alone, without the need to request such information from the athlete. Accordingly, the system 300 provides a particularly advantageous measurement system that allows for the measurement of subjective characteristics of an athlete based on the objectively measured kinematic data from a pod 102. Such technology provides a substantial technological advance in measurement techniques for athletes.

While the above example is described for analyzing real-time kinematic data with the ML model 326, the ML model 326 may also process kinematic data after the kinematic data has been collected for an athletic activity, such as for a post-run analysis. In such examples, the kinematic data may be collected for the duration of the athletic activity and then provided to the ML model 326. The ML model 326 then classifies the kinematic data (or segments thereof) with objective scores of the evaluation categories for which the ML model 326 was trained.

In the examples where the real-time kinematic data is evaluated by the ML model 326 during the athletic activity, the classifications from the ML model 326 may then be used to provide feedback or adjustments for the athlete during the athletic activity. For instance, objective scores may be used to generate an alert on the example computing device 302 to indicate to the athlete that he or she may be in poor health, suffering an injury, or other potential notifications. In other examples, the standardized classification(s) (e.g., objective score(s)) may be used to adjust a pacing strategy for the athlete. As an example, if the objective scores indicate that the athlete is suffering from an injury or negative health condition, the current recommended pace for the athlete may be reduced.

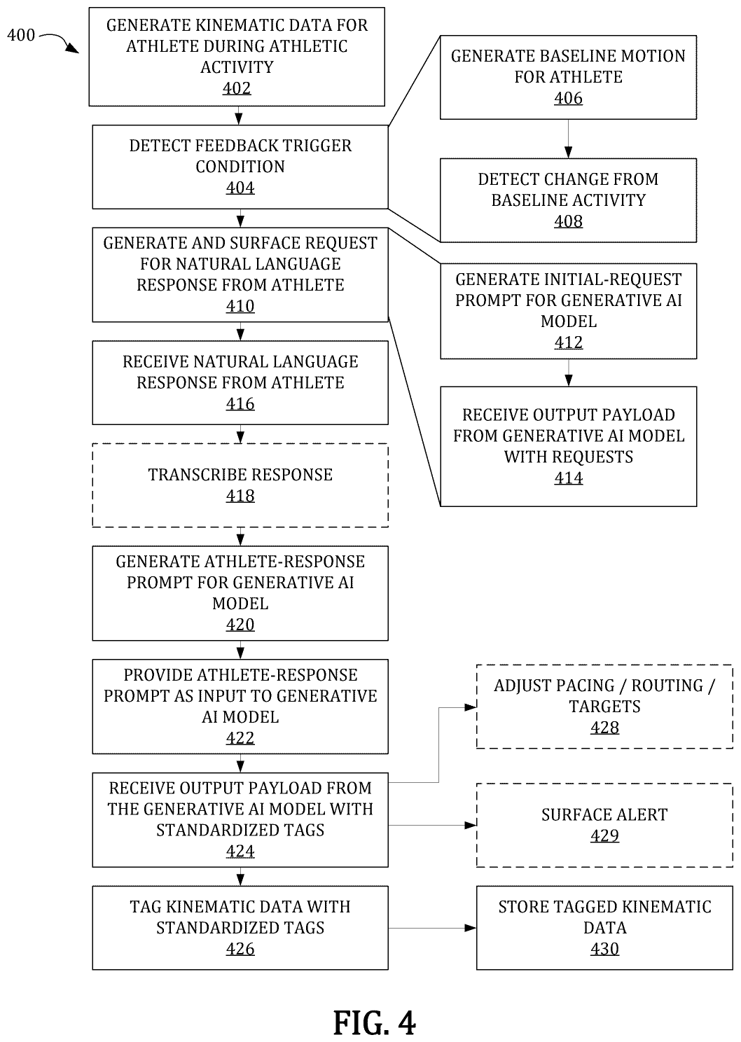

FIG. 4 depicts an example method 400 for tagging kinematic data with objective feedback data. The method 400 may be performed by the devices of the systems discussed above, such as the pod 102, measurement platform, 200 and/or the example computing device 302. Additional components of the systems may also execute some of the operations and/or be used in performing the operations of method 400.

At operation 402, kinematic data is generated for the athlete during an athletic activity (e.g., a run). The kinematic data may include any data or combinations of data from the sensors of the pod, such as the IMU and/or components thereof (e.g., accelerometers, gyroscopes, etc.). The kinematic data may include data such as velocity, acceleration, gait, ground interactions, force interactions, or other types of data that characterize the motion of the athlete and/or the limb(s) of the athlete (e.g., foot, leg).

At operation 404, a feedback trigger condition is detected. Detection of the feedback trigger condition indicates that feedback should be requested from the athlete. Different forms of feedback trigger conditions may be used in operation 402. In some examples, the feedback trigger condition may be based on a time interval (e.g., every N number of minutes or a particular percentage of a set workout routine). In other examples, the feedback trigger condition may be a distance that has been traveled by the athlete (e.g., every 2 miles, 5 miles). The feedback trigger condition may also be based on a particular interval (e.g., time and/or distance) of a particular activity. In some examples, the trigger condition may also be based on a user input received at the portable computing device.

In other examples, the feedback trigger condition may be dynamic and based on a diversion or change from a baseline in the athlete's motion. In such examples, operation 404 further comprises operations 406 and 408. At operation 406, a baseline motion for the athlete is generated or accessed. The baseline motion may be for the particular athletic activity that is being performed by the athlete. The baseline motion may be a particular pattern of motion (e.g., gait pattern) along with other types of kinematic data that are indicative of baseline motion for the athlete. The baseline motion may also be for a particular grade and speed. For example, an athlete running slowly uphill will have a different baseline gait pattern than an athlete running quickly downhill. The baseline motion that is generated or accessed for the athlete may have been generated from prior athletic activities and/or the current activity. The baseline motion may also be updated or adjusted during the athletic activity.

At operation 408, a divergence from the baseline is motion is detected. The divergence from the baseline motion may be determined by detecting that the particular motion associated baseline motion (e.g., gait pattern), for the current speed and grade, has changed by a threshold amount. In some examples the divergence from the baseline motion may be detected through a mathematical comparison of the baseline kinematic data to the current kinematic data. In other examples, the divergence may be detected through the use of a classifier (e.g., an ML model) that classifies segments of the kinematic data as matching or diverging from the baseline motion for the current speed and grade. In yet other examples, such a divergence may be detected by providing the current kinematic data and the baseline motion as input to the generative AI model and requesting the generative AI model to determine if the current kinematic data diverges from the baseline motion.

At operation 410, one or more requests for a natural language response from the athlete are generated during the athletic activity. These feedback requests may be generated from locally stored requests, as discussed above. In other examples, the feedback requests may be generated through the use of a generative AI model. In such examples, operations 412-414 are performed.

At operation 412, an initial-request prompt is generated. The initial-request prompt may include the content and details discussed above. The initial-request prompt is then provided as input to the generative AI model, which processes the initial-request prompt. The generative AI model generates an output payload that includes that feedback request(s). The output payload is received at operation 414.

As part of operation 410, the feedback request(s) are surfaced to the athlete during the athletic activity. As discussed above, the feedback requests may be surfaced in a variety of manners, such as via an audio output (e.g., through a speaker or headphones) and/or visual output (such as on a display screen).

At operation 416, the natural language response is received from the athlete. The natural language response may be received in an audio format via a microphone of the computing device or through a microphone of connected headphones. In other examples, the natural language response may be received via text input into the computing device, such as via keyboard or touch screen. At operation 418, for examples where the natural language input is received as a voice or audio input, the voice input may be transcribed into text using a speech-to-text algorithm. The received natural language response is then correlated with the corresponding feedback request that solicited the natural language response.

At operation 420, an athlete-response prompt is generated that incorporates the natural language response(s) to the feedback request(s). The athlete-response prompt may include the details discussed above. For instance, the athlete-response prompt includes an instruction to generate standardized tags for specific categories for the natural language responses. The standardized tags may be quantified tags (e.g., objective scores).

At operation 422, the athlete-response prompt is provided as input to the generative AI model. The generative AI model then processes the athlete-response prompt to generate an output payload that includes the standardized tags. At operation 424, that output payload is received and the standardized tags are extracted from the output payload.

In some examples, actions can be taken based on the standardized tags that are received (e.g., based on the objective scores). For instance, operation 428 may be performed where a pacing value, routing, and/or other targeting is adjusted for the athlete. In some examples, the system generates a pacing value for the athlete that provides a recommended pace for the athlete to achieve the goals of the athlete. This pacing value may be adjusted dynamically based on the standardized tags. For example, if the standardized tags indicate that the athlete is currently suffering an injury, the pacing value may be changed to a slower pace to accommodate for the injury and prevent further injury or aggravation of the injury. Alternatively or additionally, the routing information may be adjusted. For example, if a particular route is set for the athlete for a current run, that route may be dynamically adjusted based on the standardized tags. For instance, if the standardized tags indicate tat the athlete is currently suffering an injury or illness, the route may be shortened to a shorter route. In examples where there are multiple segments to a current athletic activity or workout, the targets and/or durations for the current and/or subsequent segments may be adjusted based on the standardized tags. Future planned workouts or plans may also be adjusted automatically based on the standardized tags, such as an increasing risk for injury or illness. In some examples, the actions may be taken automatically, or an alert or prompt may be surfaced to the athlete requested confirmation that the athlete wants to make the recommended changes. The alert may further include options for selection by the athlete for different actions that may be taken.

In some examples, also based on the standardized tags, at operation 429, an alert may be surfaced to the athlete via an audible, visual, or haptic notification. The alert may indicate to the athlete that may be in poor health, suffering an injury, or other potential notifications based on the standardized classification of the kinematic data. The alert may also relate to form and/or cadence of the athlete. For instance, particular form suggestions may be generated based on the standardized tags. The form suggestions may provide suggestions for changes in how the athlete is running that would allow the user to continue to achieve the targets for the run, such as the pacing target. As an example, based on the standardized tags, a particular injury or ailment may be identified which may be alleviated based on a change in form of running (or otherwise performing the athletic activity).

At operation 426, kinematic data corresponding to the natural language responses are tagged with the standardized tags received in operation 424. As discussed above, the kinematic data corresponding to the natural language responses may be kinematic data that received a set amount of time before, or before and after, the natural language responses were received.

At operation 430, the tagged kinematic data is stored. For instance, the tagged kinematic data may be transmitted to a remote or cloud database for storage.

Method 400 may then repeat for multiple athletic activities of the athlete and/or athletic activities of multiple athletes. As such, large amounts of tagged kinematic data may be generated and stored within the database and be used for pattern recognition and training of machine learning models, as discussed further herein.

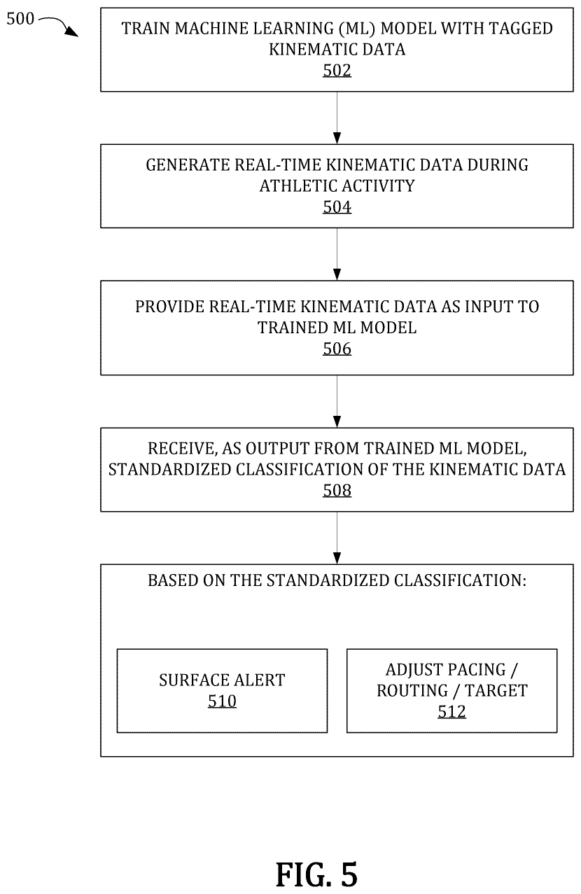

FIG. 5 depicts an example method 500 for classifying kinematic data. The operations of method 500 may be performed by the systems and components thereof described above.

At operation 502, an ML model is trained with the tagged kinematic data, such as through supervised training techniques. Supervised training uses the tagged kinematic data as the labeled training dataset, with the kinematic data being treated as the input and the standardized tags being treated as the known output or ground-truth output. During training, the weights of the ML model 326 may be iteratively adjusted based on the tagged kinematic data until the ML model 326 is able to accurately predict or classify the kinematic data to match the standardized tags (within an accuracy tolerance). Operation 502 may be performed at an interval basis at a non-runtime period (e.g., late night periods or other periods where minimal traffic is experienced). Operation 502 may also be performed by a remote device (e.g., cloud server) that hosts or has access to the tagged kinematic data stored in the kinematic database.

Once the ML model is trained, the ML model may be accessed and used via a network (e.g., internet connection). In other examples, the trained ML model may be loaded on to the personal computing device (e.g., example computing device 302) or the pod for local processing of data by the trained ML model. For instance, the ML model may be of a substantially smaller size, and require substantially fewer processing resources, than the generative AI model. As such, the trained ML model may be stored and executed locally on much smaller devices with fewer computing resources (e.g., pod, smart watch, smart phone).

As discussed above, the ML model may be trained to be athlete specific. In such cases, the ML model may be trained primarily or predominately on the tagged kinematic data from the athlete. In other examples, the ML model may be more generic or trained for groups of athletes that share one or more characteristics.

At operation 504, real-time kinematic data is generated during an athletic activity. The kinematic data may include any data or combinations of data from the sensors of the pod, such as the IMU and/or components thereof (e.g., accelerometers, gyroscopes, etc.). The kinematic data may include data such as velocity, acceleration, gait, ground interactions, force interactions, or other types of data that characterize the motion of the athlete and/or the limb(s) of the athlete (e.g., foot, leg).

At operation 506, the real-time kinematic data is provided as input into the trained ML model. The trained ML model processes the real-time kinematic data to generate an output that includes a standardized classification of the kinematic data. That output is received in operation 508. In some examples where the ML model is executing locally, the output is received locally. In other examples, the output may be received via an internet connection or other network connection.

The standardized classification corresponds to one or more of the standardized tags of the kinematic data used to train the ML model. For instance, the standardized classification may be one or more objective scores of one or more of the categories discussed herein. As examples, the classifications may provide a classification related to health, injury, fatigue, hydration, weather, athletic equipment (e.g., shoes), or mood, among other categories. For instance, the classification may provide a severity or score for one or more of the categories, such as a severe likelihood for injury, shoes that may not be an optimal choice, and/or increased fatigue state, among others.

Based on the standardized classification for the kinematic data, one or more of operations 510 or 512 may be performed. The operations 510 and 512 may be similar to and include the same or similar types of adjustments and alerts as operations 428 and 429 discussed above. At operation 510, an alert may be surfaced to the athlete via an audible, visual, or haptic notification. The alert may indicate to the athlete that he or she may be in poor health, suffering an injury, or other potential notifications based on the standardized classification of the kinematic data. In some examples, the alert may also request that that the athlete provide a confirmation (or rejection) of the condition set forth in the alert. For example, if the alert indicates that the athlete is likely suffering from an injury, the athlete may be requested to confirm or deny the existence of such an injury. The response from the athlete may then be collected from the device and used for reinforcement training of the ML model.

At operation 512, a real-time pacing target, routing, and/or other targets for the athlete may be adjusted. In some examples, the system generates a pacing target for the athlete that provides a recommended pace for the athlete to achieve the goals of the athlete. This pacing target may be adjusted dynamically based on the standardized tags. For example, if the standardized tags indicate that the athlete is currently suffering an injury, health issue, poor nutrition or hydration, the pacing target may be changed to a slower pace to accommodate for condition of the athlete. The pacing target may also be adjusted based on other factors, such as measurements from the sensors of the measurement platform 200.

Operations 504-512 of method 500 may repeat for multiple segments or intervals of the athletic activity to assess the condition of the athlete via the standardized classifications of the kinematic data for the particular segment or interval. Operations 504-521 may also be repeated for additional athletic activities for the athlete or other athletes.

Aspects of the present invention, for example, are described above with reference to block diagrams and/or operational illustrations of methods, systems, and computer program products according to aspects of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved. In some examples, not all operations may be performed. Further, as used herein and in the claims, the phrase “at least one of element A, element B, or element C” is intended to convey any of: element A, element B, element C, elements A and B, elements A and C, elements B and C, and elements A, B, and C.

The description and illustration of one or more examples provided in this application are not intended to limit or restrict the scope of the invention as claimed in any way. The aspects, examples, and details provided in this application are considered sufficient to convey possession and enable others to make and use the best mode of claimed invention. The claimed invention should not be construed as being limited to any aspect, example, or detail provided in this application. Regardless of whether shown and described in combination or separately, the various features (both structural and methodological) are intended to be selectively included or omitted to produce an example with a particular set of features. Having been provided with the description and illustration of the present application, one skilled in the art may envision variations, modifications, and alternate examples falling within the spirit of the broader aspects of the general inventive concept embodied in this application that do not depart from the broader scope of the claimed invention.

We claim:

1. A measurement system that is retained by an athlete during an athletic activity, the measurement system comprising:
a housing that includes an accelerometer and a gyroscope;
at least one processor; and
at least one memory, the at least one memory storing instructions that, when executed by the at least one processor cause the system to perform operations comprising:
generate kinematic data for the athlete during the athletic activity based on measurements from the accelerometer and the gyroscope;
detect an occurrence of a feedback trigger condition;
based on the occurrence of the feedback trigger condition, surface a feedback request for a natural language response from the athlete during the athletic activity;
receive, in response to the feedback request, a natural language response from the athlete during the athletic activity;
generate an athlete-response prompt for a generative artificial intelligence (AI) model, the athlete-response prompt including the natural language response and instructions to transform the natural language response to at least one standardized tag for one or more defined categories;
provide the athlete-response prompt as input to the generative AI model;
receive, from the generative AI model in response to the athlete-response prompt, an output payload includes the at least one standardized tag for the one or more defined categories; and
tag the kinematic data with the at least one standardized tag for the one or more defined categories to create tagged kinematic data.

2. The measurement system of claim 1, wherein the operations further comprise train a machine learning (ML) model the tagged kinematic data.

3. The measurement system of claim 2, wherein the kinematic data is first kinematic data and the athletic activity is a first athletic activity, and the operations further comprise:
generate second kinematic data for the athlete during a second athletic activity based on measurements from the accelerometer and the gyroscope;
provide the second kinematic data as input to the trained ML model during the second athletic activity; and
receive, as output from the trained ML model during the second athletic activity, a standardized classification of the kinematic data.

4. The measurement system of claim 3, wherein the operations further comprise, based on the standardized classification of the kinematic data, surface a notification to the athlete.

5. The measurement system of claim 3, wherein the operations further comprise, based on the standardized classification of the kinematic data, adjusting at least one of a pacing target or routing target for the athlete during the second athletic activity.

6. The measurement system of claim 1, wherein the operations further comprise, based on the standardized tag, adjusting a pacing target for the athlete during the athletic activity.

7. The measurement system of claim 1, wherein the operations further comprise generating the feedback request by:
generate an initial-request prompt instructing the generative AI model to generate the feedback request;
provide the initial-request prompt as input to the generative AI model; and
receive, from the generative AI model in response to the initial-request prompt, an output payload with the feedback request.

8. The measurement system of claim 7, wherein the initial-request prompt includes at least one of user-profile data for the athlete or the kinematic data.

9. A computer-implemented measurement method for measuring kinematic data of an athlete during athletic activities, the method comprising:
generating kinematic data for the athlete during an athletic activity based on measurements from at least one of an accelerometer and a gyroscope;
detecting an occurrence of a feedback trigger condition;
based on the occurrence of the feedback trigger condition, surface a feedback request for a natural language response from the athlete during the athletic activity;
receiving, in response to the feedback request, a natural language response from the athlete during the athletic activity;
generating an athlete-response prompt for a generative artificial intelligence (AI) model, the athlete-response prompt including the natural language response and instructions to transform the natural language response to at least one standardized tag for one or more defined categories;
providing the athlete-response prompt as input to the generative AI model;
receiving, from the generative AI model in response to the athlete-response prompt, an output payload includes the at least one standardized tag for the one or more defined categories; and
tagging the kinematic data with the at least one standardized tag for the one or more defined categories to create tagged kinematic data.

10. The computer-implemented method of claim 9, further comprising, based on the standardized tag, performing at least one of:
adjusting a pacing target for the athlete during the athletic activity; or
surfacing a notification to the athlete.

11. The computer-implemented method of claim 9, wherein the kinematic data is first kinematic data and the athletic activity is a first athletic activity, and the method further comprises:
training a machine learning (ML) model the tagged kinematic data;
generating second kinematic data for the athlete during a second athletic activity based on measurements from the accelerometer and the gyroscope;
providing the second kinematic data as input to the trained ML model during the second athletic activity; and
receiving, as output from the trained ML model during the second athletic activity, a standardized classification of the kinematic data.

12. The computer-implemented method of claim 11, further comprising, based on the standardized classification, adjusting a pacing target for the athlete during the second athletic activity.

13. The computer-implemented method of claim 11, wherein the standardized classification is for at least one of a health category or an injury category.

14. The computer-implemented method of claim 13, further comprising, based on the standardized classification, surfacing an alert to the athlete indicating an injury or health condition of the athlete.

15. The computer-implemented method of claim 9, further comprising generating the feedback request by:
generating an initial-request prompt instructing the generative AI model to generate the feedback request;
providing the initial-request prompt as input to the generative AI model; and
receiving, from the generative AI model in response to the initial-request prompt, an output payload with the feedback request.

16. The computer-implemented method of claim 9, wherein the generating the kinematic data and detecting the occurrence of a feedback trigger condition are performed by a foot pod housing the accelerometer and the gyroscope.

17. A computer-implemented measurement method for measuring kinematic data of an athlete during athletic activities, the method comprising:
generating kinematic data for the athlete during an athletic activity based on measurements from at least one of an accelerometer and a gyroscope;
providing the kinematic data at input to a trained machine learning (ML) model, wherein the trained ML model is trained based on prior kinematic data tagged with standardized tags generated from a generative artificial intelligence (AI) model based on natural language responses received from one or more athletes;
receiving, as output from the trained ML model during the athletic activity, a standardized classification of the kinematic data; and
based the standardized classification adjusting a target for the athlete during the athletic activity.

18. The computer-implemented method of claim 17, wherein the standardized classification indicates the athlete is suffering at least one of an injury or a health condition, and adjusting the target adjusts a pacing target to a slower pace.

19. The computer-implemented method of claim 17, further comprising, based on the standardized classification, surfacing an alert to the athlete.

20. The computer-implemented method of claim 19, further comprising:
receiving, in response to the alert, and input from the athlete; and
performing a reinforcement training of the ML model based on the input from the athlete.
